(19) United States
(12) Patent Application Publication
(10) Pub. No.: US 2022/0364991 A1
(43) Pub. Date: Nov. 17, 2022

(54) Method and System for Decoding Information Stored on a Polymer Sequence

(71) Applicant: Northeastern University, Boston, MA (US)

(72) Inventors: Meni Wanunu, Sharon, MA (US); Fatemeh Farhangdoust, Allston, MA (US)

(21) Appl. No.: 17/703,933

(22) Filed: Mar. 24, 2022

Related U.S. Application Data
(60) Provisional application No. 63/165,415, filed on Mar. 24, 2021.

(51) Int. Cl. G01N 21/64; G16B 50/00; C12Q 1/68

(52) U.S. Cl. CPC G01N 21/6428; G16B 50/00; C12Q 1/68; G01N 21/6458; G01N 2021/6432; G01N 2021/6439

(57) ABSTRACT

A method and system to decode information stored on a polymer sequence, such as a DNA strand, is described herein. The method and system use molecular probes to label sections of the polymer sequence. Each molecular probe includes a fluorophore and a quencher. The fluorophore produces light with a color and wavelength corresponding to the information stored on the section of the polymer sequence the molecular probe labels. The quencher inhibits the production of light by an adjacent fluorophore. When adjacent sections of the polymer sequence are labeled with molecular probes, the fluorophore of the leading molecular probe produces light while the trailing molecular probe's light is quenched. The method and system then sequentially unbind the molecular probes from the sections of the polymer sequence within a waveguide, producing a sequence of observable fluorescence signals. The sequence can be used to determine the information stored on a polymer sequence.

### RELATED APPLICATION

This application claims the benefit of U.S. Provisional Application No. 63/165,415, filed on Mar. 24, 2021. The entire teachings of the above application are incorporated herein by reference.

### GOVERNMENT SUPPORT

This invention was made with government support under Grant Number R01HG011087 awarded by the National Institutes of Health. The government has certain rights in the invention.

### BACKGROUND

As technology progresses, there is an ever increasing need to store greater amounts of data and information. Conventional storage media like flash-drives and hard-drives do not have the longevity, data density, or cost efficiency to meet this demand. One alternative to conventional storage media is to store data on DNA strands or other polymers, where the sequence or pattern of the component molecules, e.g., nucleotides, corresponds to encoded information. This storage method provides orders of magnitude greater density of information than convention storage media.

Once information is encoded onto DNA strands or other polymers, the information needs to be decoded, and the data represented by the sequence of the component molecules needs to be transformed back into a usable format, usually binary data. Methods exist, such as polymerase chain reaction, for sequencing DNA strands or other polymers, so that the pattern of their component molecules can be determined. This determined pattern can then be interpreted as the stored data. However, there are many limitations in existing methods, such as i) their slow speed in sequencing the DNA strands or other polymer, ii) the required time needed to interpret and convert the pattern of their component molecules into stored data, and iii) the difficulty dealing with a range of sizes of the DNA strands or other polymer. A need exists for a method of rapidly decoding information stored on DNA strands and/or other polymers of various sizes.

### SUMMARY

An example embodiment of the invention is an optical method for reading out blocks or sections of a polymer chain, such as DNA, rather than individual bases, to decode a polymer chain at a single molecule level, thereby increasing a rate of readout.

A method for decoding information stored on a polymer sequence comprises unbinding labels from a polymer sequence in a sequential manner, a given label attached to the polymer sequence at a corresponding pattern of component molecules that correspond to a portion of information encoded into the polymer sequence. The method further includes observing a sequence of fluorescence signals produced by unbinding the labels and decoding the information encoded into the polymer sequence based on the sequence of fluorescence signals observed.

In some embodiments, the method begins by attaching the labels to the polymer sequence. The labels may be molecular probes that have a leading end and a trailing end defined relative to a direction travel of the polymer sequence while unbinding of the labels occurs. The molecular probe includes a fluorophore at the leading end that emits a fluorescence signal at a wavelength based on the corresponding pattern of component molecules and a quencher that inhibits a fluorescence signal emitted by an adjacent fluorophore at a leading end of a trailing adjacent molecular probe.

The polymer sequence may be one of a DNA strand, a synthetic polymer, or a synthetic biopolymer. The corresponding pattern of component molecules can comprise a segment of the polymer sequence and in such embodiments, the information encoded into the polymer sequence is encoded as a pattern of the segments. The portion of information encoded into the polymer sequence may be a binary n-bit value, wherein n=2 or integer multiple thereof.

The unbinding labels from the polymer sequence in the sequential manner can be performed by unwinding the polymer sequence by use of an enzyme. The decoding the information encoded into the polymer sequence based on the observing of the sequence of fluorescence signals can also be performed in real-time.

The method may be performed in a fluidic cell that includes a fluid and defines a nanowell, and the method further includes applying a voltage in the fluidic cell that produces an electric field in the fluid, causing the polymer sequence to be drawn toward an observation region of the nanowell. In such embodiments, the applied voltage can be further configured to draw unlabeled portions of the polymer sequence away from the observation region of the nanowell.

A system for decoding information stored on a polymer sequence comprising a nanowell with an observation region and an enzyme configured to unbind labels from a polymer sequence in a sequential manner at the observation region. A given label is attached to the polymer sequence at a corresponding pattern of component molecules that correspond to a portion of information encoded into the polymer sequence. The system also includes a sensor configured to observe a sequence of fluorescence signals produced by unbinding the labels in the observation region and a processor communicatively coupled to the sensor and configured to decode the information encoded into the polymer sequence based on the sequence of fluorescence signals observed.

The nanowell can be a zero-mode waveguide or an electrochemically actuatable zero-mode waveguide. The nanowell may include an electrode of an electrode pair, the electrode pair configured to apply a voltage that produces an electric field in the fluid that causes the polymer sequence to be drawn toward the observation region of the nanowell. The include electrode may be a platinum layer underneath the observation region.

The system can further include a channel in fluidic communication with the observation region of the nanowell and that is configured to enable transport of unlabeled portions of the polymer sequence away from the observation region.

The polymer sequence of the system may be a DNA strand, a synthetic polymer, or a synthetic biopolymer. The labels can be molecular probes. In such embodiments, the molecular probes have a leading end and a trailing end defined relative to a direction of travel of the polymer sequence while unbinding of the labels occurs and the molecular probe include a fluorophore at the leading end that emits fluorescence light at a wavelength based on the corresponding pattern of component molecules and a quencher that inhibits fluorescence light emitted by an adjacent fluorophore at a leading end of an adjacent trailing molecular probe.

The sensor may be a fluorescence microscope.

The system may also include a fluidic cell that defines the nanowell, contains an electrolyte solution therein, and is configured to receive the polymer sequence. The nanowell may be defined by at least one boundary surface that is a transparent element and wherein the sensor is arranged to observe the fluorescence signal through the transparent element.

### BRIEF DESCRIPTION OF THE DRAWINGS

The patent or application file contains at least one drawing executed in color. Copies of this patent or patent application publication with color drawings will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A is a flow diagram of a method for decoding data encoded on a polymer sequence utilized by embodiments of the invention.

FIG. 1B is an illustration of a molecular probe utilized by embodiments of the invention.

FIG. 1C is an illustration of molecular probes, attached in series to a polymer sequence, utilized by embodiments of the invention.

FIG. 2A is an illustration of an array of electro-optical zero-mode waveguides (eZMWs) utilized by embodiments of the invention.

FIG. 2B is an illustration of a single electro-optical zero-mode waveguide of the array shown in FIG. 2A.

FIG. 2C is a photograph of a 1×1 cm eZMW chip containing four eZMW arrays.

FIG. 2D is a diagram that shows the output of an emCCD camera used to detect light produced in eZMW wells in an embodiment of the invention.

FIG. 2E is an illustration of a fluidic cell that holds an array utilized by embodiments of the invention.

FIG. 2F is magnified sections of the diagram that shows the output-of an emCCD camera shown in FIG. 2D.

FIG. 3A is a scanning electron microscopy (SEM) image of a 3×9 subset of a 36×106 eZMW array.

FIG. 3B is a transmission electron microscopy (TEM) image cross-sectional view of an eZMW well.

FIG. 3C is a zoomed-in section of the transmission electron microscopy (TEM) image of FIG. 3B.

FIG. 3D is a high-resolution, energy-dispersive. X-ray spectroscopy (EDS) map of the eZMW well sample shown in FIGS. 3B and 3C.

FIG. 4A is a graph of a comparison of intensity decay profile between an eZMW with 40 nm-thick Al₂O₃ and a standard aluminum-based zero mode waveguide (AlZMW) with a single 100 nm-thick Al layer for light with wavelengths of 532 nm and 640 nm.

FIG. 4B is a graph of the comparison of field enhancement capabilities for eZMWs with 10, 40, 80, and 160 nm thick Al₂O₃ layer and a standard AlZMW.

FIGS. 4C and 4D are 3D finite-element simulations of intensity distribution (log scale) for an eZMW with 40 nm Al₂O₃ layer at 532 nm and 640 nm, respectively.

FIG. 5A is a graph of the I-V characteristics of an eZMW array versus platinum electrode for different voltage biases during 5 voltage-sweep cycles at a scan rate of 10 mV/s in 10×10⁻³M KCl.

FIG. 5B is a graph of the I-V characteristics of an eZMW array versus platinum electrode for different voltage biases during 5 voltage-sweep cycles at a scan rate of 10 mV/s in 10×10⁻³ M sequencing buffer.

FIG. 6A is a diagram of frame-integrated images of loading 500 pb DiYO-labeled DNA into eZMWs 201 after 10, 20, and 80 s of 100 mV voltage application.

FIG. 6B is a graph of the loading percentage over time for different length DNA strands.

FIG. 6C is a graph of the loading percentage over time for different concentration DNA strands.

FIG. 6D is a graph of the loading percentage over time for different applied voltages.

FIG. 7A is cross-sectional side view of an electro-optical zero-mode waveguide (eZMW) with an unwinding enzyme according to an example embodiment of the present invention.

FIG. 7B is a fluorescence signal produced by an embodiment of the invention.

FIG. 7C is a diagram of the positioning of molecular probe, that produces green light, and molecular probe, that produces red light, during a red fluorescence burst.

FIG. 7D is a diagram of the positioning of molecular probe, that produces green light, and molecular probe, that produces red light, during a green fluorescence burst.

FIG. 8 is cross-sectional side view of an array with two eZMWs with an underground channel according to another example embodiment of the present invention.

FIG. 9 is a flow chart of a method of decoding data stored on a polymer sequence according to an embodiment of the invention.

### DETAILED DESCRIPTION

A description of example embodiments follows.

Embodiments of the invention include a method for capturing and decoding a polymer sequence using sequence-encoded optical molecular probes attached to a long polymer sequence. The sequence-encoded optical molecular probes, or molecular probes, attach to wound polymer sequence segments and are later unattached from polymer sequence segments. Unattached molecular probes quickly diffuse. The unwinding of a polymer sequence with sequence-encoded optical molecular probes attached produces a fluorescence signal that corresponds to the pattern of the component molecules of the polymer sequence that in turn corresponds to the encoded data.

Embodiments of invention enable fast readout of information stored on polymer sequences and enable high throughput achieved via parallel detection from multiple waveguides unwinding polymer sequences. Embodiments of the invention also use highly processive enzymes that work for a very long time to facilitate the unwinding. Furthermore, some embodiments of the invention also include an ability to sort molecules post-readout using channels below the waveguides.

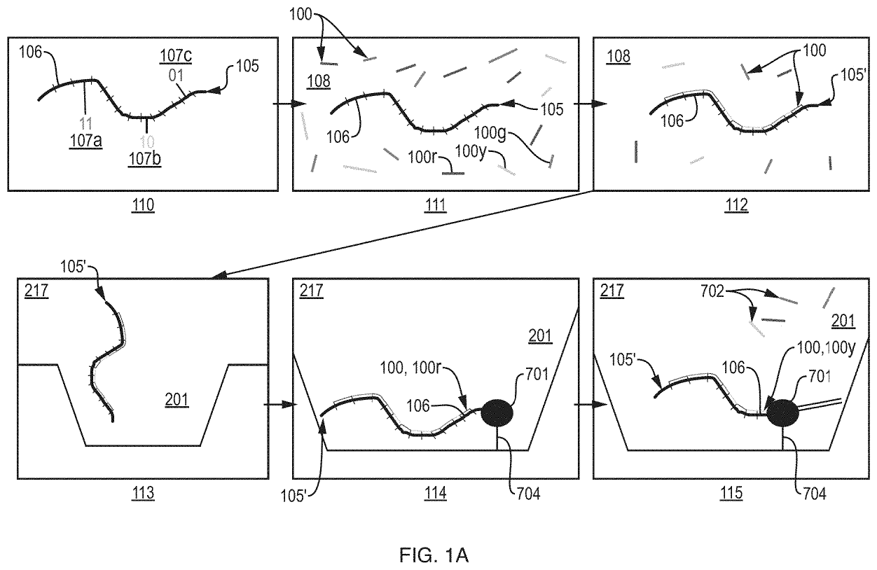

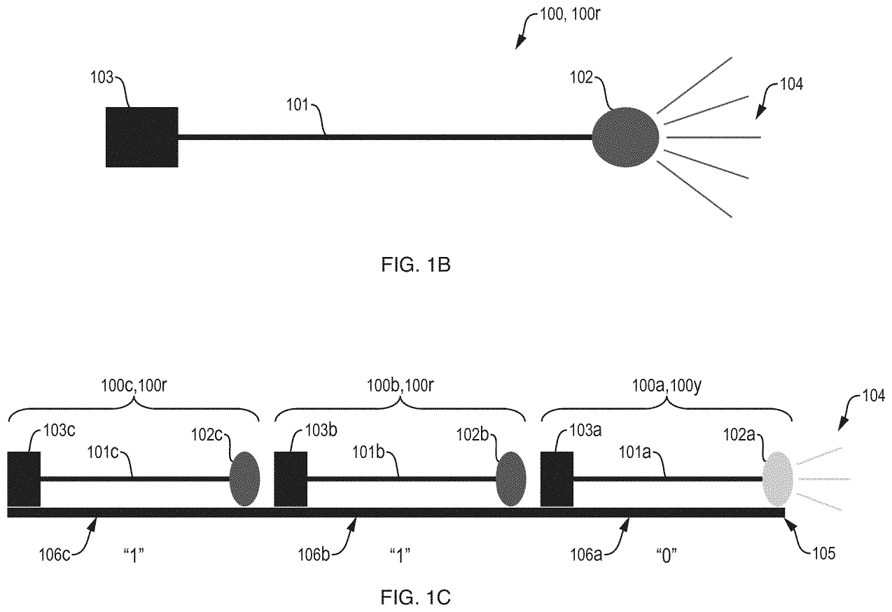

FIG. 1A is a flow diagram of a method for decoding data, also referred to herein as “information,” encoded on a polymer sequence utilized by embodiments of the invention. In step 110, a polymer sequence 105 is received. The polymer sequence 105 has data 107a (e.g., binary data “11”), 107b (e.g., binary data “10”), 107c (e.g., binary data “01”) encoded onto its sections 106, within which portions of data or information are contained, where the portions may be any length, sequence, or other characteristic. For example, the data 107a, 107b, and 107c are portions of the data encoded onto all of the polymer sequence 105.

The encoding may be performed by correlating the pattern of the component molecules of sections 106 with respective encoded data 107a, 107b, 107c. A polymer sequence 105 can be comprised of a series of sections 106, each section 106 encoded with data 107a, 107b, 107c. Multiple segments 106 may include an identical pattern of component molecules with identical pieces of data encoded thereon. Therefore, it should be understood that the data encoded onto the polymer sequence 105 can also be encoded as a pattern of segments 106 with each segment 106 representing a different portion of data 107a, 107b, 107c.

In step 111, labels, for example molecular probes or probes 100, such as probes 100r, 100g, 100y, are added into a solution 108 with the polymer sequence 105. Molecular probes 100 are shown in more detail in FIGS. 1B and 1C and described in their associated description below. In this example embodiment, the molecular probes 100, e.g., 100r, 100g, 100y, are configured to emit different colors, in this case, red, green, and yellow. The emitted colors are correlated to the section 106, and its pattern of component molecules, at which the respective molecular probes 100, 100r, 100g, 100y bind. For example, a probe 100g configured to bind to a section 106 encoded with data “11” 107a emits green light; a probe 100y configured to bind to a section 106 encoded with data “10” 107b emits yellow light; and a probe 100r configured to bind to a section 106 encoded with data “01” 107c emits red light. The light may be a fluorescence light or may be any other type of visible or non-visible signal that is observable by an appropriate sensor (not shown), such as instrumentation that can convert a visible or non-visible signal produced by a probe into a digital representation thereof.

In step 112, the probes 100, 100r, 100g, 100y bind to sections 106. A probe 100r, for example, will bind to a section 106 with a specific pattern of component molecules. This results in a labeled polymer sequence 105′ having a series of probes 100 bound to it. The colors emitted by the probes 100, 100r, 100g, 100y during a process of sequential unlabeling will correspond to the sequential pattern of molecules of the sections 106 of the polymer sequence 105′ that correspond to the encoded data 107a, 107b, and 107c. If the number of probes 100 exceeds the number of sections 106, some probes 100 will remain in the solution 108 and unbound to the polymer sequence 105′. After a labeled polymer sequence 105′ is created, if the color of light produced by the series of bound probes 100 can be determined, it can be used to decode the encoded data 107a, 107b, and 107c.

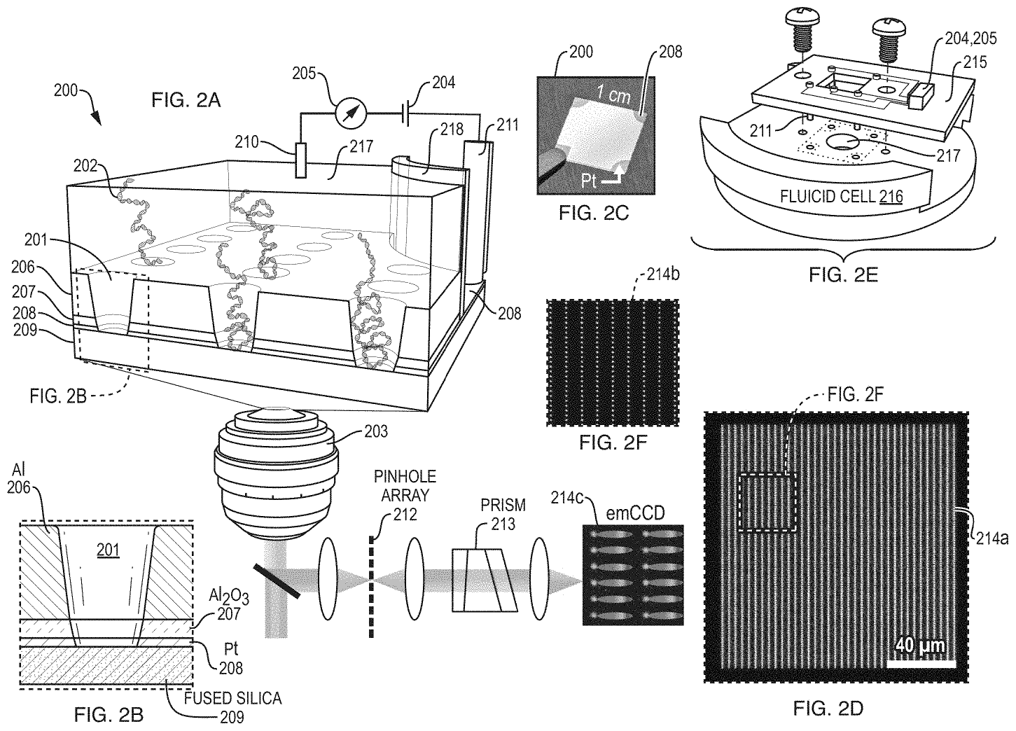

In step 113, the labeled polymer sequence 105′ is placed in a fluidic cell 216 containing an electrolyte solution 217 and loaded into a waveguide 201 on the fluidic cell's 216 bottom surface (detailed in FIGS. 2A and 2B). Loading may be accomplished through diffusion of the polymer sequence 105′ or by the application of a voltage gradient to draw the labeled polymer sequence 105′ electronically into the waveguide 201. As will be described in reference to the steps 114 and 115, the waveguide 201 enables detection of light, at its color/wavelength, emitted by the probes 100, 100r, 100g, 100y.

In step 114, after the labeled polymer sequence 105′ is loaded into a waveguide 201, an enzyme 701, tethered with linker 704 to the waveguide 201, is activated. The enzyme 704 begins to unwind labeled polymer sequence 105′ inside the waveguide 201. In some embodiments, unwound parts of the labeled polymer sequence 105′ are directed into a channel away from the waveguide 201. Because of the component elements of molecular probes 100, 100r, 100g, 100y (described in further detail below), only the probe 100, 100r attached to the section 106 at the leading end of labeled polymer sequence 105′, closest to enzyme 701, can produce detectable light. Therefore, in the example shown in step 114, only red light, corresponding to data “01” 107c, is produced and detected inside the waveguide 201.

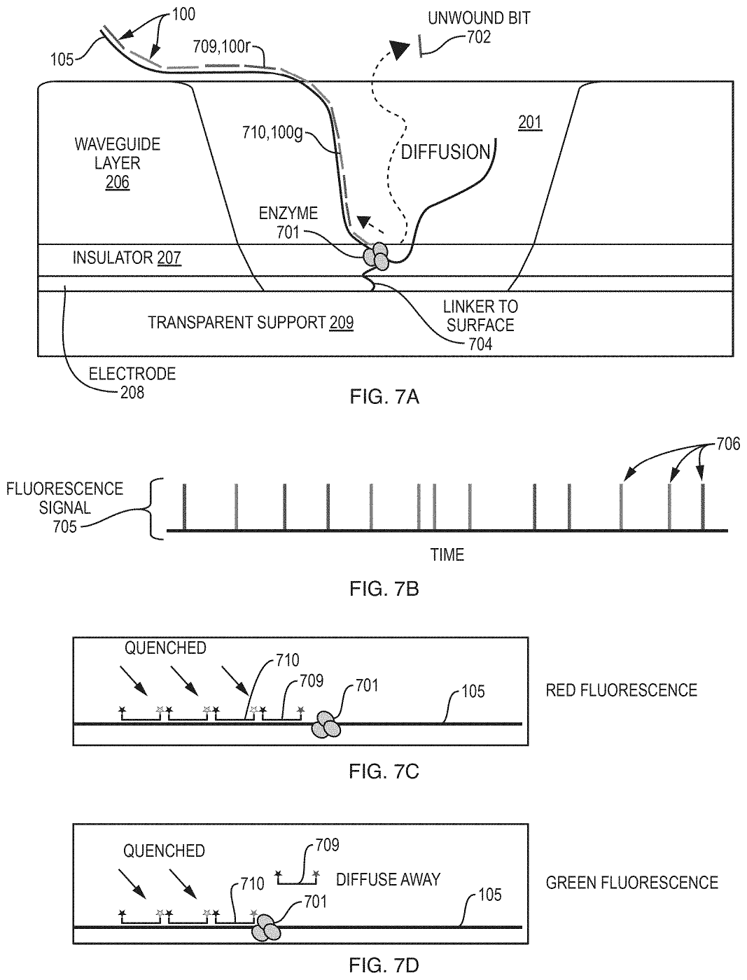

As shown in step 115, an enzyme 704 unwinds the labeled polymer sequence 105′. After a section 106 is unwound, its bound molecular probe detaches and diffuses 702. A diffused molecular probe 702 no longer produces any detectable light. Therefore, as the labeled polymer sequence 105′ is unwound, the molecular probes 100 detach and diffuse in series, sequentially exposing a new section 106 and attached probe 100, for example in step 115 a 100y probe is now exposed, that emits light, e.g. yellow light. This process results in the creation of a sequence of fluorescence signals within the waveguide 201. The sequence of fluorescence signals, composed of a series of light bursts emitted by the probes 100, 100r, 100g, 100y, continues as each probe becomes the leading probe as the preceding probe detaches and defuses 702. The sequence of fluorescence signals corresponds to and can be used to identify the sections 106 that comprise the polymer sequence 105, 105′. If the portions of data 107a, 107b, 107c that each section 106 has encoded on its pattern of component molecules is known, then the sequence of fluorescent signals, by providing the identity of the sections 106, can be used to decode the encoded data 107a, 107b, 107c. Steps 114 and 115 are shown in more detail in FIGS. 7A-7D.

A processor that is part of or in communication with a computer, with a memory storing the correlation between color/wavelength of light produced by probes 100, 100r, 100g, 100y and identity of sections 106 and/or the correlation between the identity of sections 106 and the encoded data 107a, 107b, 107c, can be used to translate, in real-time, the observed fluorescence signal inside waveguide 201 into the encoded data 107a, 107b, 107c.

FIG. 1B is an illustration of a molecular probe 100, 100r utilized by embodiments of the invention. The molecular probe 100, 100r includes fluorophore 102 that emits light 104 and a quencher 103. Because fluorophore 102 emits red light 104, the shown molecular probe is a red light emitting probe 100r. The quencher 103 inhibits the emission of light by an adjacent fluorophore 102. Different fluorophores 102 can be used by different molecular probes 100 that emit different colors/wavelengths of light 104. The fluorophore 102 and quencher 103 are connected to and separated by a body 101. The body 101 is configured to attached to a segment of a polymer sequence so that the fluorophore 102 and quencher 103 are located at the start and end of the segment. The body 101 can be configured to attach only to a segment 106 of a polymer sequence 105 that has a particular pattern of component molecules that corresponds to a portion of encoded information. By using different fluorophores 102 (e.g. red, yellow, and green) for molecular probes 100 (e.g. 100r, 100y, and 100g) configured to attached to different segments 106 with different patterns of component molecules that correspond to different portions of encoded information, the light 104 emitted by fluorophores 102 can be used to identify the pattern of component molecules, or sequence, of the segment 106 the body 101 attaches to. In turn, the identified pattern of component molecules, or sequence, can be used to determine the portion of information encoded on that segment. In other words, the light 104 emitted by fluorophore 102 is correlated with and can be used to determine the encoded information in a labeled segment 106. Importantly, because the molecular probe 100, 100r, 100y, 100g is configured to attached to and label a segment 106 of a polymer sequence 105, the emitted light 104 corresponds and provides information regarding the pattern of component molecules of the entire segment 106 of a polymer sequence 105 and not just single component molecules.

FIG. 1C is an illustration of molecular probes 100, attached in series to a polymer sequence 105, utilized by embodiments of the invention. Molecular probes 100a (a yellow emitting probe 100y), 100b (a red emitting probe 100r), and 100c (a red emitting probe 100r) are attached to polymer sequence 105. The bodies 101a, 101b, and 101c of molecular probes 100a, 100b, and 100c are configured to attached to segments 106a, 106b, and 106c respectively. Molecular probes 100a, 100b, and 100c have fluorophores 102a, 102b, 102c and quenchers 103a, 103b, 103c. When in series, the quenchers 103a, 103b, 103c block the light emitted from the fluorophores 102a, 102b, 102c of the following molecular probes 100a, 100b, and 100c. Therefore, only the molecular probe 103a at the front of the series can emit light 104 from its fluorophore 102a.

Segments 106b and 106c have the same pattern of component molecules and therefore store the same encoded information, for example a “1” used in binary. Therefore, molecular probes 100b, 100r and 100c, 100r have the same fluorophores 102b, 102c, that produce the same color light (red), and the same bodies 101b, 101c configured to attached to segments 106b and 106c composed of the same pattern of component molecules. Segment 106a has a different pattern of component molecules and therefore stores different encoded information, for example a “0” used in binary. Therefore, molecular probe 100a, 100y has a different fluorophore 102a, that produces different color light (yellow), and a different body 101a configured to attach to segment 106a and any other similar segment (not shown). If the series of molecular probes 100a, 100b, and 100c are viewed as displayed in FIG. 1C, only light from fluorophore 102a will be observed as light from fluorophores 102b, 102c are blocked by quenchers 103a, 103b. If molecular probes 100a is removed, for example due to diffusion after segment 106a is unwound, quencher 103a no longer blocks light emitted by fluorophore 102b and it will be able to be observed.

If molecule probes 100a, 100b, 100c are removed in sequence, for example by segmentally unwinding their corresponding labeled sections 106a, 106b, and 106c, a series of light will be observed produced by fluorophore 102a, then fluorophore 102b, and then fluorophore 102c. The pattern of light, will correspond to the pattern of segments 106a, 106b, and 106c and the information encoded within them. Therefore, an observed light pattern of “yellow”, “red”, “red” corresponds to and can be used to decode the encoded information “0”, “1”, “1” stored on polymer sequence 105. FIG. 1C illustrate an embodiment with two colors of fluorophore 102a, 102b, 102c that correspond to two pieces of encoded data. A person of skill in the art should understand that many colors of fluorophores 102a, 102b, 102c can be used that correspond to an equivalent number of pieces of encoded data. One benefit of this method of decoding stored information, is that the attached molecular probes 100a, 100b, 100c can be removed and the series of emitted light observed in real time allowing the information encoded in segments 106a, 106b, and 106c to also be interpreted in real time.

Using an example of DNA, a DNA strand can be decoded in this method by the sequential unwinding of fluorescently-labeled oligonucleotides such that an order of fluorescence bursts reports on contents of a corresponding DNA sequence that can correlated to stored information. Producing only a single fluorescence signal from only the last bit of unwound the DNA sequence is achieved by coupling, using an attached molecular probe 100, a fluorophore 102 on one end of the oligonucleotide and a quencher 103 on the other end, such that oligonucleotides are only fluorescent when there is no quencher 103 near the fluorophore 102 component. Using multi-color single-molecule fluorescence, one can decode the contents of a DNA strand based on a time-series of fluorescence colors produced. Each color would be utilized by a different probe 100 configured to attach to a different sequence of oligonucleotides. Therefore, the observed color of the fluorescence bursts corresponds to the sequence of the DNA strand. The time-series of fluorescence colors can be produced by unwinding the DNA strand in segments with each unwound segment corresponding to the length of the attached probes 100. After each segment is unwound, a new probe 100 would be located at the end of the wound DNA strand and the probe's 100 fluorophore 102 would no longer be blocked by the proceeding probe's 100 quencher 103 as the proceeding probe 100 would be unattached and diffused when its segment was unwound.

FIG. 2A is an illustration of an array 200 of electro-optical zero-mode waveguides (eZMWs) 201 that can be utilized by embodiments of the invention. Electro-optical zero-mode waveguides (eZMWs) 201 can be used to observe the time-series of fluorescence colors produced by fluorophores 102 of molecular probes 100 that correlate to and can be used to decode the information stored on segments 106 of polymer sequence 105, 202. Multiple eZMWs 201 can be combined into an array 200. The polymer sequences 105, 202 are drawn into eZMW(s) 201 by an applied voltage of voltage source 204 controlled and measured by ammeter 205. Voltage application across the eZMW(s) 201 leads to ion flow to embodied electrodes at their base that allows voltage-induced capture of polymer sequences such as DNA strands. This eliminates the requirement for a freestanding ultrathin membrane that forms the porous base of such as nanopore ZMWs (NZMWs) and porous ZMWs (PZMWs), improving stability and reducing background optical noise. In some embodiments, the eZMW(s) 201 are 100 nm diameter nanowells.

FIG. 2B is an illustration of a single electro-optical zero-mode waveguides of the array shown in FIG. 2A. The array 200, may have a base of a fused silica chip 209 that contains multiple eZMWs 201 wells. The fused silica chip 209 is transparent and allows light produced within eZMWs 201 to be observed from below the array 200. Embodiments can use electro-optical zero-mode waveguide 201 wells that comprises an aluminum (Al) cladding layer 206 (top) that serves to generate a zeromode light confinement effect. Underneath the Al layer, there is a thin dielectric alumina (Al₂O₃) layer 207 that serves as an insulating layer, and below it is a thin (<10 nm) Platinum-disk electrode 208 which serves as a working electrode for application of electric fields within the eZMW 201. Upon voltage application, from source 204 between the platinum disk and a current-carrying wire 210 (shown in FIG. 2A), such as platinum wire, placed in the flowcell chamber that contains electrolyte solution 217 and the eZMW 201, a strong localized electric field is created from local Faradaic reactions in the buffer within the eZMW 201. This localized field allows for the efficient electrokinetic capture of DNA/RNA molecules, and other polymer sequences 105, 202, that diffuse to the proximity of the eZMW 201. After the polymer sequences 105, 202 are captured by eZMW 201, they can be unwound and produce the time-series of fluorescence colors used to decode the information stored.

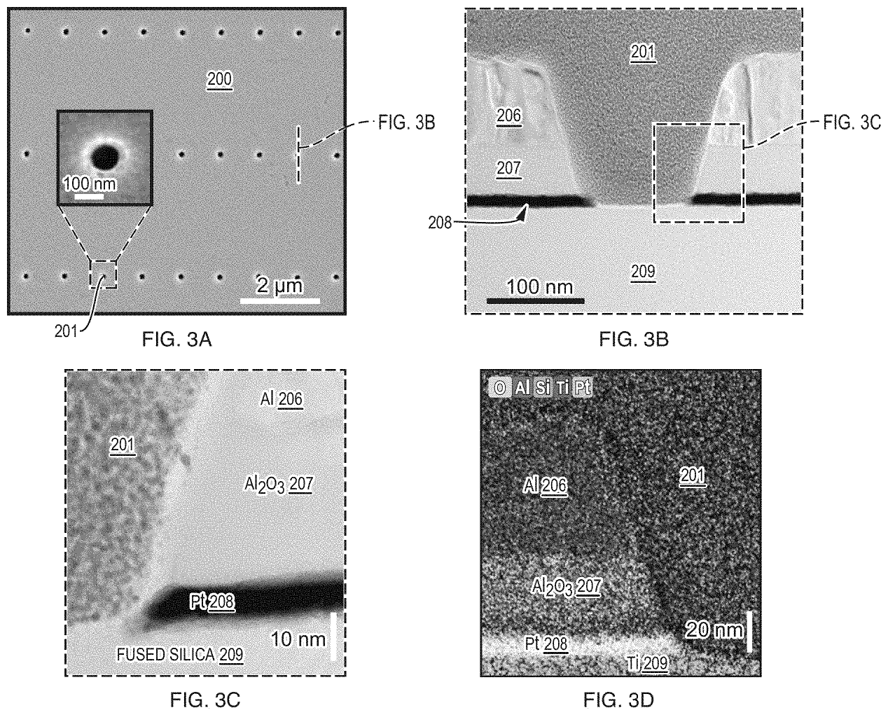

FIG. 2C is a photograph of a 1×1 cm eZMW chip that contains four arrays 200 containing eZMWs 201 wells. Each array 200 has 36×106 (3816 in total) eZMWs 201 wells. Therefore, each eZMW chip has approximately 15000 eZMWs 201 wells. Each eZMWs 201 well is able to decode the information on a DNA strand and each eZMWs 201 in an array or chip can operate is parallel. The Platinum (Pt) layer 208 is exposed at the corners of the array 200 to facilitate electric contact. To expose the Platinum (Pt) layer 208, the Al 206 and Al₂O₃ 207 layers may be etched using a standard photolithography process. The exposed Pt layer is isolated from the rest of the array 200 using SU-8 to prevent liquid contact. The Returning to FIG. 3A, pin 211 is shown contacting Pt layer 208, incorporating it into a circuit with voltage source 204, ammeter and controller 205, and wire 210 isolated by SU-8 218.

A sensor, for example fluorescence microscope 203, is located below array 200 and can detect light in the eZMWs 201 wells through the fused silica 209 or other transparent element base. In some embodiments, the image captured by fluorescence microscope 203 is transmitted through a pinhole array 212 and a prism 213, that provides the angular dispersion required for detection of all color fluorescence light produced by fluorophores 102. The produced light and resulting time series can also be detected using an electron-multiplying charge-coupled device (emCCD) camera. The sensor, such has fluorescence microscope 203 or a emCCD camera, can be connected to a processor or computer that is able determine the detected light in the eZMWs 201 well and use it to decode to data stored on polymer sequence 105. The processor or computer may have or be in communication with a memory that stores the correlation between the produced light's color/wavelength and a pattern of component molecules representing portions of encoded data.

FIG. 2D shows the output 214a of an emCCD camera used to detect light produced in eZMWs 201 wells in an embodiment of the invention. FIG. 2F shows magnified sections 214b and 214c of the output 214a of an emCCD camera used to detect light produced in eZMWs 201 wells shown in FIG. 2D. Each dot in the output 214a, corresponds to light produced in an individual eZMWs 201 of array 200. As the polymer sequence 105, 202 is unwound and different fluorophores 102 are uncovered by quenchers 103, the light produced and displayed on output 214 will change. The color of the produced and displayed on output 214 will correlate to the pattern of molecules that comprise a segment 106 of the polymer sequence 105, 202 within eZMWs 201 wells. Therefore, the colors produced and displayed on output 214 can be used to decode information stored on the pattern of molecules of the polymer sequence 105, 202. The observed produced light allows for the pattern of component molecules, and the encoded data, to be determined in real-time.

FIG. 2E is an illustration of a fluidic cell 216 that holds array 200. Array 200 can be secured onto fluidic cell 216 by chip 215. The fluidic cell 216 can be a poly(ether ether ketone) (PEEK) fluidic cell that configured to hold the array 200 on its bottom surface. Chip 215 includes circuit components used to generate the localized electric field, such as voltage source 204 and ammeter and controller 205. Fluidic cell 216 holds an electrolyte solution 217 into which polymer sequences 105, 202 can be diffused and drawn into eZMWs 201 wells. Pins 211 are connected to the circuit on chip 215 and to the Pt layer 208 of array 200. Pins 11 can be spring-loaded pogo pins that have been soldered to allow electrical connection to a patch clamp amplifier (Axopatch 200B) for voltage application between the Pt layer 208 and the wire 211 present in the sample chamber of fluid cell 216 above eZMWs 201 wells of array 200. Fluidic cell 216 includes a view hole 217 to enable the fluorescence microscope 203 to capture the light produced in the eZMWs 201 wells.

eZMWs 201 utilized by embodiments of the invention can be fabricated on UV-grade 170 μm-thick fused silica wafers via standard electron-beam lithography, layer-by-layer deposition, and lift-off methods. Briefly, a negative tone e-beam resist is spun-coated on a wafer, followed by scanning a focused beam of electrons to make patterns corresponding to the eZMW array 200. This results in nanopillars remaining on the wafer, which is then deposited with successive layers of Ti, Pt, Al₂O₃, and Al using an e-beam evaporator. Next, the resist pillars along with the metal caps are dissolved leaving behind nanoapertures, the imprint of the pillars. Then, photolithography is performed on the aluminum side of the wafer to expose only the four corners that will be etched to provide access to the Pt layer 208, followed by etching of the Al 206 and Al₂O₃ 207 layers. Finally, the wafers with exposed Pt areas are divided into individual 1 cm×1 cm squares (see FIG. 2C).

FIG. 3A is a scanning electron microscopy (SEM) image of a 3×9 subset of a 36×106 eZMW array 200. In array 200, the eZMW wells 201 ZMWs are spaced 1.33 μm (short axis) and 4.0 μm (long axis). The bright outline around each eZMW 201 is an artifact that stems from its slanted shape that forms due to a shadowing effect from the pillars during the metal deposition process.

FIG. 3B is a transmission electron microscopy (TEM) image cross-sectional view of an eZMW 201 well. FIG. 3C is a zoomed in section of the transmission electron microscopy (TEM) image of FIG. 3B. FIGS. 3B and 3C were generated by focused ion beam (FIB) milling a thin lamella and transferring it to a transmission electron microscopy (TEM) grid. FIGS. 3B and 3C show the tapered waveguide structure of eZMW 201 well with a top diameter of with a diameter 200±10 nm and a bottom diameter of 100±10 nm. FIGS. 3B and 3C also show the layers of eZMW 201 well clearly, the (Al) cladding layer 206, the thin dielectric alumina (Al₂O₃) layer 207 and the platinum-disk electrode 208 on top of fused silica 209.

FIG. 3D is a high-resolution energy-dispersive X-ray spectroscopy (EDS) map of the eZMW 201 well sample shown in FIGS. 3B and 3C. The elements that comprise the layers of the eZMW 201 are labeled with different colors as shown in the legend.

3D finite-element simulations of electric field intensity within 100 nm diameter eZMW 201 wells with different thicknesses of Al₂O₃ layer 207 between a 100 nm Al cladding layer 206 and an 8 nm platinum layers were performed in an overall excitation/emission range of 500-800 nm wavelengths.

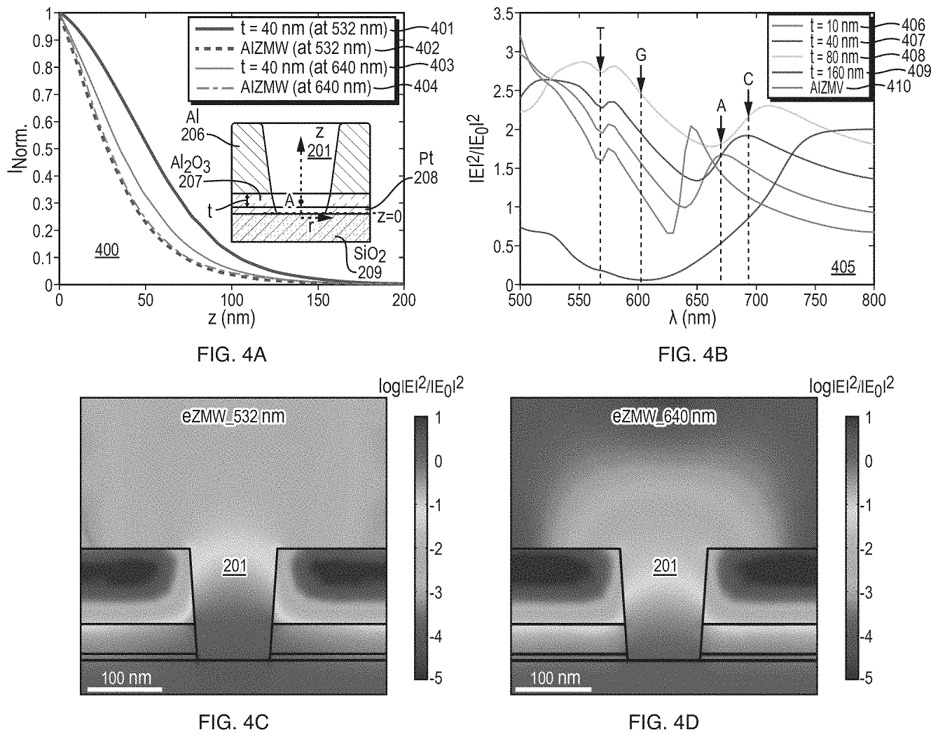

FIG. 4A is a graph 400 of a comparison of intensity decay profile between an eZMW with a 40 nm-thick Al₂O₃ layer and a standard aluminum-based waveguide (AlZMW) with a single 100 nm-thick Al layer for light with wavelengths of 532 and 640 nm. The compared eZMW 201, with the layers 206, 207, 208, and 208 is displayed in an insert. The insert also shows the location of the z-axis and the other parameters used in the simulation on a cross-sectional view of an eZMW 201. Graph 400 shows the intensity decay profile for the eZMW 201 at 532 nm 401 and 640 nm 403. Graph 400 also shows the intensity decay profile for the standard AlZMW, without an Al₂O₃ layer at 532 nm 402 and 640 nm 404. The decay profile is a measurement of the observed intensity, shown in the y-axis, of a light produced at z=0 and a distance, shown in the x-axis, from z=0. The intensity is measured and displayed as a percentage of the intensity observed at z=0. The simulation results show that the the electric field (light) intensity profile along the z-axis within the eZMW 201 shows ZMW-typical attenuation for both wavelengths, and while the characteristic decay length is not as small as for AlZMWs. Therefore, eZMWs allow for high-quality measurements and detections of the colors produced by fluorophores 102 of probes 100.

FIG. 4B is a graph 405 of the comparison of simulated field enhancement capabilities for eZMWs with 10, 40, 80, and 160 nm thick Al₂O₃ layer and a standard AlZMW. The field enhancement capacities for the 500-800 nm spectrum were measured at point A (z=10 nm, r=0) shown in the insert of FIG. 4A. Point A is the approximate position of a DNA polymerase in eZMWs 201 well and therefor the location of any colors produced by fluorophores 102 of probes 100. attached to a DNA polymerase or other polymer sequence. The results for eZMWs with 10, 40, 80, and 160 nm are shown by lines 406, 407, 408, and 409 respectively and the results for a standard AlZMW is shown by line 410. The simulation results indicate that the field at point A is higher in the layered eZMW 201 structure as compared to the case of a bare aluminum ZMW (AlZMW). In addition, the field at point A is enhanced for increasing Al₂O₃ thickness from 10 to 80 nm, with greater changes in the enhanced field in red (640 nm) wavelengths than for green (532 nm) wavelengths. Furthermore, the field is smaller at 640 nm (red), but depends much more on the Al₂O₃ thickness than for the 532 nm wavelength (green). However, while increasing the thickness of the Al₂O₃ layer to 80 nm results in larger observation volume in both red and green lasers, the overall ZMW volume increases, and therefore the amount of background from diffusing phospholinked fluorescent nucleotides is larger. Some embodiments use a thickness of 40 nm for the Al₂O₃ layer 207 considering the trade-off between increasing ZMW volumes and electrical field enhancement. Graph 405 also displays the fluorescence emission wavelength windows (as marked by T, G, A, C) of labeled nucleotides.

FIGS. 4C and 4D are 3D finite-element simulations of intensity distribution (log scale) for an eZMW with 40 nm Al₂O₃ layer at 532 nm and 640 nm respectively. FIGS. 4C and 4D show that light is attenuated greatly within the waveguide 201 for both wavelengths, and decays by 0.5 orders of magnitude within ≅z-height for 640 nm light in eZMWs of radii that range from 50 to 70 nm.

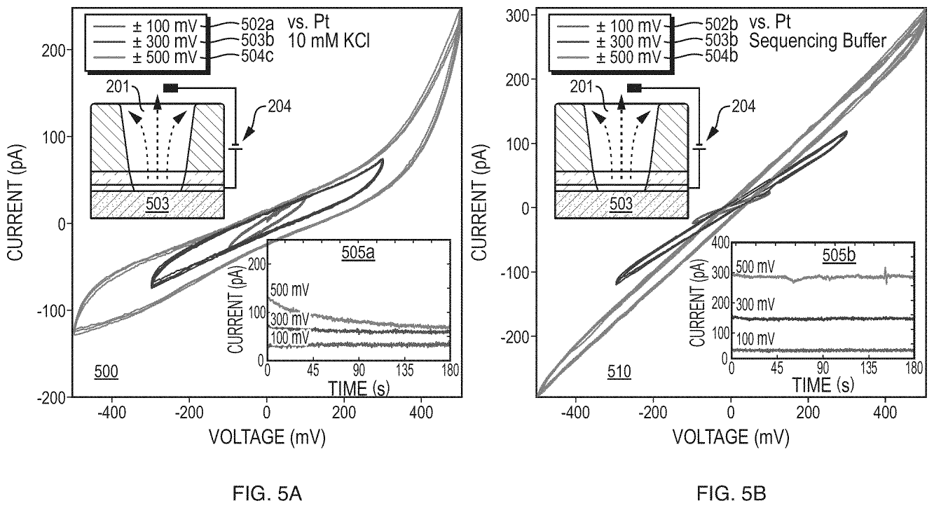

FIG. 5A is a graph 500 of the I-V characteristics of an eZMW array 200 versus platinum electrode for different voltages biases during 5 voltage-sweep cycles at scan rate of 10 mV/s in 10×10⁻³ M KCl. FIG. 5B is a graph 501 of the I-V characteristics of an eZMW array 200 versus platinum electrode for different voltage biases during 5 voltage-sweep cycles at scan rate of 10 mV/s in 10×10⁻³ M sequencing buffer. The results of an applied ±100 mV voltage are shown as lines 502a and 502b. The results of an applied ±300 mV voltage are shown as lines 503a and 503b. The results of an applied ±500 mV voltage are shown as lines 504a and 504b. The voltages are applied to eZMW 201 wells of array 200 by voltage source 204 shown in inserts of FIGS. 5A and 5B and in more detail in FIG. 2A. The recorded I-V curves for the 10×10⁻³ M KCl buffer are displayed on graph 500, which represent 5 successive voltage sweep cycles, exhibit a highly symmetrical hysteresis behavior caused by capacitance at the electrode/solution interface. By contrast, this hysteresis behavior is weaker and the current magnitude higher in the results for the sequencing buffer displayed on graph 501, due to a higher extend of Faradaic reactions produced by redox-active agent, nitrobenzoic acid (NBA) in the sequencing buffer. In all cases, the voltage sweeps are reproducible and overlapping, indicating electrochemical stability of the eZMWs 201 and array 200. In addition, the eZMW 201 wells were inspected to ensure that the current produced, displayed in FIGS. 5A and 5B, was not due to aluminum corrosion.

The inserts 505a and 505b are graphs of typical current-time (I-t) trances of an eZMW in 10×10⁻³ M KCl 505a and sequencing buffer 505b for different voltages. In contrast to the case of pure KCl electrolyte, the sequencing buffer, which contains a redox-active species NBA, produces a steady current level that hints on Faradaic processes at the electrodes. When measuring I-V curves at different voltage scan rates, the anodic and cathodic peak currents are to be linearly dependent on the scan rate in the sequencing buffer for all voltages (R²>0.99 for all fits). This indicates that a diffusion-control process exists on the Pt electrodes 208, and its contribution is greater than the capacitive current according to the Randels-Sevcik equation. Other solutions, such as 5×10⁻³ M K₃Fe(CN)₆ and 5×10⁻³ M K₄Fe(CN)₆ with 10×10⁻³ Ms aqueous KCl supporting electrolytes, and other alternative solutions can also be utilized by embodiments of the invention.

eZMWs 201, utilized by embodiments of the invention, are designed to overcome the high input DNA requirements and length biases of diffusion-based loading. The diffusion process, naturally favors the capture of shorter DNA molecules due to the size constraints of the wells. To load and read long fragments, such as those able to store large amounts of data, size-selection systems are used that removes short fragments through gel electrophoresis. This may result in the information encoded on shorter fragments being lost. Additionally, large amounts of DNA amounts (3>μg per 1 Gb genome) will be needed decreasing efficiency and the density of stored information.

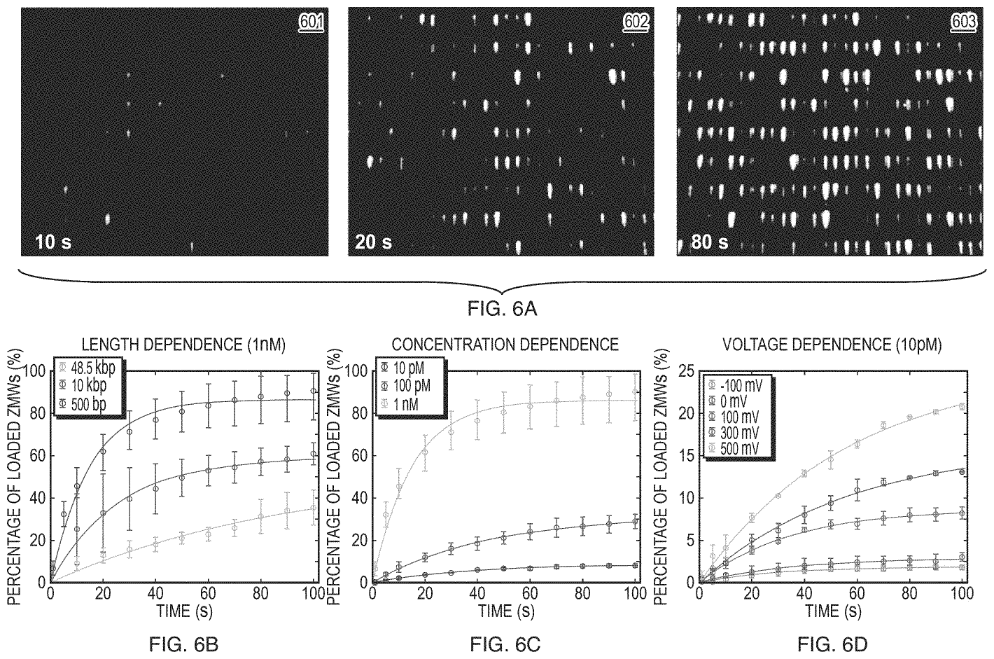

FIG. 6A is frame-integrated images of loading 500 bp DiYO-labeled DNA into eZMWs 201 after 10 (image 601), 20 (image 602), and 80 (image 603) s of 100 mV voltage application. FIG. 6A is composed of integrated fluorescence snapshots of an array 200 containing eZMWs 201 with a 1×10⁻⁹ m 500 bp DNA solution at ±100 mV applied voltage. When an eZMWs 201 is loaded with a DNA strand, it produces light due to exciting by a blue laser (488 nm) the fluorescently labeled double-stranded DNA. The number of active eZMWs, loaded with DNA, rises over time which indicates to a time-stable capture of DNA molecules inside the eZMWs.

FIG. 6B is a graph 604 of the loading percentage over time for different length DNA strands. Graph 604 shows the loading percentage over time for 48.5 kbp, 10 kbp and 500 bp long DNA strands. Graph 604 shows an approximately threefold increased loading efficiency from 500 bp NDA, as compared with 48.5 kbp DNA. This is a relatively insignificant bias considering the order of magnitude larger radius of gyration of 48.5 kbp DNA (≅500 nm) as compared to 500 bp DNA (≅nm).

FIG. 6C is a graph of the loading percentage over time for different concentration DNA strands. Graph 605 shows the loading percentage over time for 10 pM, 100 pM, and 1 nM DNA strand concentration. DNA loading is concentration dependent, with initial loading rates approximately proportional to the concentrations.

FIG. 6D is a graph 606 of the loading percentage over time for different applied voltages. Graph 606 shows the loading percentage over time for of 500 bp DNA under −100 mV, 0 mV (diffusion loading only), 100 mV, 300 mV, and 500 mV applied voltages. Increasing the voltage has a drastic effect on loading rates and greatly promotes DNA capture. Additionally, negative voltage repels DNA from the eZMWs more effectively than no voltage. To capture the data of graph 606, a localized field was created (within hundreds of nanometers from the eZMW 201 “mouth”), sufficient to capture/trap DNA molecules present in the vicinity. This simulates the field created by applying voltage using voltage source 204 and the rest of the circuit depicted in FIG. 2.

As described above, the eZMWs 201 are an effective novel device for electrically capturing DNA, or other polymer sequences, into wells. Multiple eZMWs 201 can be combined to form a single array 200 on a solid fused silica substrate 209. The eZMWs 201 are able the capture DNA, or other polymer sequences, and confine any light they, or attached probes 100, produce. Both short and long fragments can be captured with high efficiency and relatively low length bias. In addition, the eZMWs 201 allow for low input DNA capture and analysis without any amplification steps.

eZMWs 201 can be used for sequencing of individual component molecules of polymer sequences, e.g. DNA sequencing. But, sequencing is often too slow and expensive for storing information, sometimes the information units are blocks of nucleotides linked together. An example embodiment of the invention allows readout of those blocks without having to sequence every base in the DNA, or component molecules of other polymer sequences (faster and more efficient). If polymer sequence is labeled using molecular probes 100, shown in FIGS. 1B and 1C, a detailed analysis of the individual component molecules is unneeded. Instead, segments 106 of the polymer sequence 105 can be identified based on the light 104 produced by fluorophore 102. The pattern of the component molecules of segments 106 is already known, as well as the correlation between the color/wavelength of light 104 produced by fluorophore 102 and the labeled segment. Therefore, by observing the light 104 produced by fluorophore 102 the identify of segment 106 can be known and therefore the pattern of its component molecule and any information represented by that pattern without analyzing each individual component molecule.

When a polymer sequence 105 labeled with molecular probes 100 is first captured by an eZMW 201, due to quenchers 103, only the front end fluorophore 102 will produce light 104 that provides information about front end segment 106. Therefore, molecular probes 100 must be removed from polymer sequence 105 in a controllable and predictable pattern to reveal more fluorophores 102 that will produce light 104 that can be used to identify additional segments 106. Embodiments of the invention use enzymes that unwind polymer sequence 105. When a segment 106 is unwound, its attached molecular probe 100, detaches and defuses exposing the fluorophore 102 of the next molecular probe 100. As the polymer sequence 105 is unwound by an enzyme within a eZMW 201, a series of florescent bursts will be produced by the sequential exposure of fluorophores 102 of molecular probes 100. This series of florescent will correspond to the identity of segments 106 and the information encoded within their component molecules.

FIG. 7A is cross-sectional side view of an electro-optical zero-mode waveguide (eZMW) 201 with an unwinding enzyme 701 according to an example embodiment of the present invention. Embodiments of the invention use electro-optical zero-mode waveguide (eZMW) 201 with a waveguide layer 206 (e.g. Al), insulator layer 207 (e.g. Al₂O₃), electrode 208 (e.g. Pt), and transparent support 209 (e.g. fused silica). The layers 206, 207, 208, and 209 of eZMW 201 can be in the forms discussed previously but one of ordinary skill in the art would understand that alternative forms and materials could also be untitled.

A polymer sequence 105 (e.g. DNA) is labeled with molecular probes 100 with quenchers 103 and fluorophores 102, as shown in FIG. 1C. Some molecular probes 100 emit red light 709, 100r and are configured to bind to a first pattern of component molecules of the polymer sequence 105. Other molecular probes 100 emit green light 710, 100g and are configured to bind to a second pattern of component molecules of the polymer sequence 105. The polymer sequence 105, e.g. duplex DNA, is loaded into eZMW 201 either due to diffusion or electrically due to an applied voltage and field. One example embodiment employs the concept of electrically drawing DNA molecules into the eZMWs 201 using an electrode layer 208 at the bottom of the eZMW 201. This can be achieved by either applying a DC voltage differential between a solution above an insulator layer 207 at the bottom of the well and the electrode 208, or by applying an AC or pulsed field to achieve dielectrophoresis. In the dielectrophoresis approach, size-selection can be achieved where DNA, or other polymer, fragments of different lengths can be drawn into the wells selectively. Voltage can also be used to remove the DNA from the eZMWs, as well as to draw molecules in different directions after their readout.

Enzyme 701 is attached to the surface of eZMW 201 with a linker molecule 704. The linker molecule 704 allows a, for example a DNA helicase enzyme, to be immobilized while retaining its enzymatic activity. The enzyme 701 chemically functionalized eZMW 201 and in some embodiments, is a DNA helicase enzyme that unwinds sequentially unwinds segments of a DNA strand. In this non-limiting example, the polymer sequence 105 is a DNA strand that has a particular (known) sequence is hybridized to a series of 4-5 oligonucleotides in various permutations of their order. The series of oligonucleotides have corresponding molecular probes 100 (e.g. 100r, 100g) denoted in FIG. 7A by different colors, that have fluorophores 102 that produce different wavelengths of light (e.g. red and green). The molecular probes 100 are configured to attached and label their corresponding oligonucleotides series.

The series of oligonucleotides, and therefore their corresponding molecular probes 100 can be arranged in 4⁴ or 4⁵ unique combinations. The specific combination of these series of oligonucleotides corresponds to encoded data and can be programmed by choosing the correct DNA template. The length of each of these oligonucleotides may be in the range of 10-30 nucleotides, and these will be labeled, by the corresponding molecular probes 100, at their 3′ and 5′ ends with a fluorophore 102 and a quencher 103 in order to quench the fluorescence of the oligonucleotides that is adjacent to the last oligonucleotides in the sequence. In this manner (for the non-limiting example case of the 10-mer sequence), every 40 nucleotides in the DNA sequence 105 represents one binary 8-bit value of encoded data.

Typical helicase enzyme 701 unwinding rates are 10-200 nucleotides per second, so unwinding of one 8-bit should take under a second. As the DNA sequence 105 is unwound, molecular probes 100 detach and unwound probes 702 diffuse. The unwound probes 702 no longer produce any light from their fluorophore 102 and their quencher 103 no longer blocks the light of the fluorophore 102 of their proceeding probe. Therefore, unwinding DNA strand 105 inside eZMW 201 produces a series of fluorescence bursts that compose a signal as the molecular probes 100 are detached in sequence. Because the colors the molecular probes 100 produce are correlated to the series of oligonucleotides, or pattern of component molecules, the colors of the series of fluorescence bursts is also correlated and can be used to identify and decode the data encoded by the series of oligonucleotides, or pattern of component molecules.

FIG. 7B is a fluorescence signal 705 produced by an embodiment of the invention. Fluorescence signal 705 is comprised of fluorescence bursts 706 that occur over time as enzyme 701 unwinds polymer sequence 105 and detaches, in series, molecular probes 100.

FIG. 7C is a diagram of the positioning of molecular probe 710, that produces green light, and molecular probe 709, that produces red light, during a red fluorescence burst 706. Molecular probe 709 is located on the furthest edge of polymer 105 not unwound by enzyme 701 and red light produced by molecular probe 709 is unquenched. Molecular probe 710 is behind molecular probe 709, so its green light is quenched by molecular probe 710. Therefore, only red light is visible during the period of time shown in FIG. 7C. However overtime, enzyme 701 unwinds polymer sequence 105 and the situation shown in FIG. 7D occurs.

FIG. 7D is a diagram of the positioning of molecular probe 710, that produces green light, and molecular probe 709, that produces red light, during a green fluorescence burst 706. Molecular probe 709 has detached and diffused away because the section of polymer sequence it was attached to was unwound by enzyme 701. Now molecular probe 710 is located on the furthest edge of polymer 105 not unwound by enzyme 701 and green light produced by molecular probe 710 is unquenched. All other molecular probes behind molecular probe 710 remain quenched and produce no light. Therefore, green red light is visible during the period of time shown in FIG. 7D. As enzyme 701 unwinds polymer sequence 105, more molecular probes 100 are detached and defused, revealing the next molecular probe in the series, producing fluorescence signal 705 comprised of fluorescence bursts 706. Because the color of fluorescence bursts 706 is correlated to the pattern of molecules of polymer sequence 105, fluorescence signal 705 can be used, in real time, to decode information stored with the pattern of molecules of polymer sequence 105.

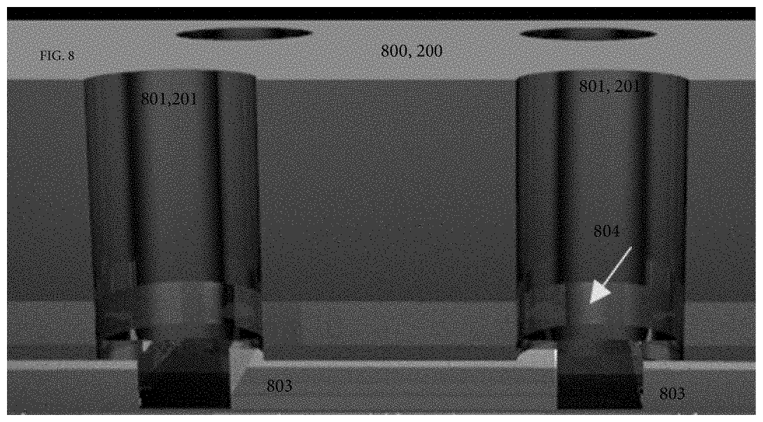

FIG. 8 is cross-sectional side view of an array 800, 200 with two eZMWs 801, 201 with an underground channel 803 according to another example embodiment of the present invention. In array 800, 200, the enzyme 701 that performs the unwinding is tethered to the sidewall 804 of the eZMW, not the bottom. This structure enables DNA, or other polymer sequences, that is unwound and detached from molecular probes to be electrically pulled downward into the underground channel 803 post-readout. This enables the DNA, or other polymer sequences, to be sorted, collected, and/or reused post readout.

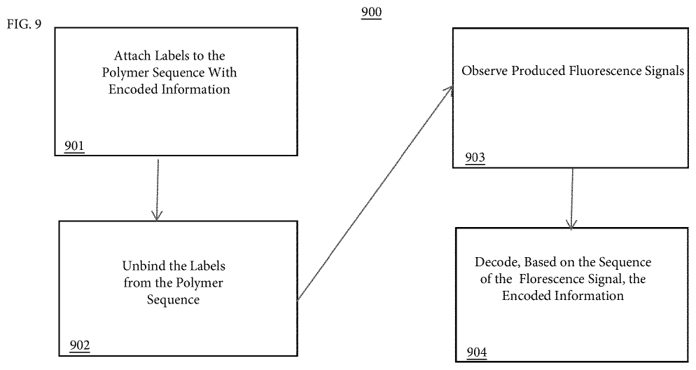

FIG. 9 is a flow chart of a method 900 of decoding data stored on a polymer sequence according to an embodiment of the invention. In the first step 901, a polymer sequence 105, for example a DNA strand, is labeled. The labels attach to the polymer sequence 105 at corresponding patterns of component molecules that that correspond to portions of information encoded into the polymer sequence 105. The labels may be molecular probes 100. In some embodiments step 901 is skipped and the method 900 starts with an already labeled polymer sequence. If the labels are molecular probes, molecular probes 100 with different fluorophores 102 are configured to attach to sections 106 of the polymer sequence 105 with different patterns of component molecules corresponding to different portions of encoded information. In that way, the fluorophores 102 and colors/wavelengths of light they emit are correlated with the patterns of component molecules of the sections 106 of the polymer sequence 105 they label. When fully labeled, the polymer sequence 105 has a linear series of molecular probes 100 attached to its linear series of segments 106. Additionally, due to the quenchers 103 of molecular probes 100, only the fluorophores 102 of the leading molecular probe 100 of the series of molecular probes 100 attached to polymer sequence 105 produces observable light.

In step 902 which occurs within a waveguide, for example an electro-optical zero-mode waveguide (eZMW) 201, the labels, such as molecular probe 100, are unbound. This may be accomplished by an enzyme 702 that sequentially unwinds segments 106 of polymer sequence 105 causing their attached molecular probe 100 labels to detach and diffuse. As the molecular probe 100 labels are unbound, a new molecular probe 100 becomes the leading molecular probe of the linear series of molecular probes 100 attached to attached to polymer sequence 105, changing the produced observable light. This change in observable light over time produces sequence of fluorescence signals.

In step 903, the waveguide is used to enable a sensor, such as a fluorescence microscope 203, to observe the produced sequence of fluorescence signals 705. Finally, in step 904, the information encoded as a pattern of the component molecules of the polymer sequence 105 is determined and decoded based the produced sequence of florescence signals 705. The sequence of fluorescence signals 705 corresponds to the linear pattern of the segments 106 comprising polymer sequence 105. Therefore, if the pattern of the component molecules of each segment 106 is known, then the fluorescence signal 705 can be used to determine the pattern of the component molecules of the polymer sequence 105. This may be done by a computer or other device able to interpret fluorescence signal 705 and/or store and apply a known correlation between the colors of fluorescence signal 705, produced by fluorophores 102 and the segments 106 of polymer sequence 105. After the pattern of the component molecules of the polymer sequence 105 is determine, any data encoded within that pattern can be decoded.

While example embodiments have been particularly shown and described, it will be understood by those skilled in the art that various changes in form and details may be made therein without departing from the scope of the embodiments encompassed by the appended claims.

What is claimed is:

1. A method for decoding information stored on a polymer sequence, the method comprising:
unbinding labels from a polymer sequence in a sequential manner, a given label attached to the polymer sequence at a corresponding pattern of component molecules that correspond to a portion of information encoded into the polymer sequence;
observing a sequence of fluorescence signals produced by unbinding the labels; and
decoding the information encoded into the polymer sequence based on the sequence of fluorescence signals observed.

2. The method of claim 1 further comprising attaching the labels to the polymer sequence.

3. The method of claim 1 wherein the labels are molecular probes, the molecular probes having a leading end and a trailing end defined relative to a direction travel of the polymer sequence while unbinding of the labels occurs, the molecular probe including a fluorophore at the leading end that emits a fluorescence signal at a wavelength based on the corresponding pattern of component molecules and a quencher that inhibits a fluorescence signal emitted by an adjacent fluorophore at a leading end of a trailing adjacent molecular probe.

4. The method of claim 1 wherein the polymer sequence is one of a DNA strand, a synthetic polymer, or a synthetic biopolymer.

5. The method of claim 1 wherein each of the corresponding pattern of component molecules comprises a segment of the polymer sequence and the information encoded into the polymer sequence is encoded as a pattern of the segments.

6. The method of claim 1 wherein the portion of information encoded into the polymer sequence is a binary n-bit value, wherein n=2 or integer multiple thereof.

7. The method of claim 1 wherein unbinding labels from the polymer sequence in the sequential manner is performed by unwinding the polymer sequence by use of an enzyme.

8. The method of claim 1 wherein decoding the information encoded into the polymer sequence based on the observing of the sequence of fluorescence signals is performed in real-time.

9. The method of claim 1 performed in a fluidic cell that includes a fluid and defines a nanowell, and wherein the method further comprises applying a voltage in the fluidic cell that produces an electric field in the fluid, causing the polymer sequence to be drawn toward an observation region of the nanowell.

10. The method of claim 9 wherein the applied voltage is further configured to draw unlabeled portions of the polymer sequence away from the observation region of the nanowell.

11. A system for decoding information stored on a polymer sequence, the system comprising:
a nanowell with an observation region;
an enzyme configured to unbind labels from a polymer sequence in a sequential manner at the observation region, a given label attached to the polymer sequence at a corresponding pattern of component molecules that correspond to a portion of information encoded into the polymer sequence;
a sensor configured to observe a sequence of fluorescence signals produced by unbinding the labels in the observation region; and
a processor communicatively coupled to the sensor and configured to decode the information encoded into the polymer sequence based on the sequence of fluorescence signals observed.

12. The system of claim 11 wherein the nanowell is a zero-mode waveguide or an electrochemically actuatable zero-mode waveguide.

13. The system of claim 11 wherein the nanowell includes an electrode of an electrode pair, the electrode pair configured to apply a voltage that produces an electric field in the fluid that causes the polymer sequence to be drawn toward the observation region of the nanowell.

14. The system of claim 13 wherein the electrode is a platinum layer underneath the observation region.

15. The system of claim 11 wherein the system further comprises a channel in fluidic communication with the observation region of the nanowell and is configured to enable transport of unlabeled portions of the polymer sequence away from the observation region.

16. The system of claim 11 wherein the polymer sequence is a DNA strand, a synthetic polymer, or a synthetic biopolymer.

17. The system of claim 11 wherein the labels are molecular probes, the molecular probes having a leading end and a trailing end defined relative to a direction of travel of the polymer sequence while unbinding of the labels occurs, the molecular probe including a fluorophore at the leading end that emits fluorescence light at a wavelength based on the corresponding pattern of component molecules and a quencher that inhibits fluorescence light emitted by an adjacent fluorophore at a leading end of an adjacent trailing molecular probe.

18. The system of claim 11 wherein the sensor is a fluorescence microscope.

19. The system of claim 11 further comprising a fluidic cell that defines the nanowell, contains an electrolyte solution therein, and is configured to receive the polymer sequence.

20. The system of claim 11 wherein the nanowell is defined by at least one boundary surface that is a transparent element and wherein the sensor is arranged to observe the fluorescence signal through the transparent element.
